(19) United States
(12) Patent Application Publication
(10) Pub. No.: US 2005/0027175 A1
(43) Pub. Date: Feb. 3, 2005

(54) Implantable biosensor

(76) Inventor: Zhongping Yang, Woodbury, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/631,908

(22) Filed: Jul. 31, 2003

(51) Int. Cl. A61B 5/00

(52) U.S. Cl. 600/302; 600/365; 600/364; 600/361; 600/309

(57) ABSTRACT

An implantable sensor includes a biosensor, integrated circuitry to operate the biosensor and an antenna to transmit data collected from the biosensor. The sensor does not include an internal power source and instead receives power from an external source in the form of RF energy. The RF energy is received by the sensor, rectified, and used as a DC source. The sensor is implanted in a subcutaneous location to allow the biosensor to measure desired characteristics.

### FIELD OF THE INVENTION

The present invention relates to sensors implantable within a human or animal body. More specifically, the present invention relates to an injectable biosensor implantable within a human or animal body and capable of translating a physiological parameter into an output signal.

### DESCRIPTION OF THE RELATED ART

Biosensors are electronic devices that produce electronic signals as the result of biological interactions. Biosensors are commonly divided into two groups. Catalytic sensors that use enzymes, microorganisms, or whole cells to catalyze a biological interaction with a target substance. Affinity systems use antibodies, receptors, nucleic acids, or other members of a binding pair to bind with a target substance, which is typically the other member of the binding pair. Biosensors are used to detect the presence and/or quantity of a giving substance within living tissue or fluids. For example, Implantable electrochemical biosensors have recently become an important tool for analyzing and quantifying the chemical composition of a patient's blood. Such biosensors are described in U.S. Published Application No. 2002/0120186, the teachings of which are incorporated herein by reference

A biosensor generally includes a sensor or biological recognition element that is placed in contact with the testable substance. An appropriate reaction occurs between the substance and the receptor that induces a measurable physical change on or within the biological recognition element. This leads to an output of the sensor in some monitorable format of an indicator in proportion to the physical change. For example, changes in potential, current flow, temperature, light output, or the like may result. These characteristics can then be output and utilized to generate data. As one example, a biosensor may be employed to monitor glucose levels. A biological recognition element may include an enzyme (glucose oxidase). When glucose contacts the enzyme, hydrogen peroxide is formed. The hydrogen peroxide produced is detected in terms of an electric signal using electrochemical means. Thus, the concentration of the substance to be detected, i.e. glucose, can be determined by detecting the amount of the resulting hydrogen peroxide.

Such a biosensor may be a self contained unit that includes a microprocessor or other dedicated circuitry from processing the data and outputting useable result. A power source, such as a battery, is required to power the circuitry. If such a biosensor is implanted, the biosensor may also include a telemetry device to transmit the data to an external source and possibly receive instructions from the external source. The telemetry device relies on an internal power source, such as the battery.

### BRIEF SUMMARY OF THE INVENTION

An implantable sensor includes a biosensor, integrated circuitry to operate the biosensor and an antenna to transmit data collected from the biosensor. The sensor does not include an internal power source and instead receives power from an external source in the form of RF energy. The RF energy is received by the sensor, rectified, and used as a DC source. The sensor is implanted in a subcutaneous location to allow the biosensor to measure desired characteristics.

In one embodiment, the present invention is an implantable sensor having a biosensor, an integrated circuit operatively coupled with the biosensor to operate and receive data from the biosensor, and a power receiver operatively coupled with the integrated circuit and configured to rectify RF energy incident on the implantable senor into DC power deliverable to the biosensor and the integrated circuit.

In another embodiment, the present invention is an implantable sensor including a biosensor, means for controlling the biosensor and means for receiving RF energy from an external source, converting the energy to DC power, and powering the implantable sensor.

In another embodiment, the present invention is a method comprising injecting a capsule containing an unpowered biosensor subcutaneously into tissue and placing an interrogator adjacent the tissue. The method further includes transmitting RF energy towards the capsule, converting the RF energy into a DC power source within the capsule, and utilizing the DC power source to power biosensor.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a schematic illustration of a biosensor and external power source according to one embodiment of the present invention.

FIG. 2 is a schematic illustration of an encapsulated biosensor.

FIG. 3 is a stylized illustration of an implantation device for delivering the encapsulated biosensor.

FIG. 4 is a stylized illustration of an implanted biosensor and an external power supply.

FIG. 5 is a flowchart illustrating a process of implanting and utilizing a biosensor.

### DETAILED DESCRIPTION

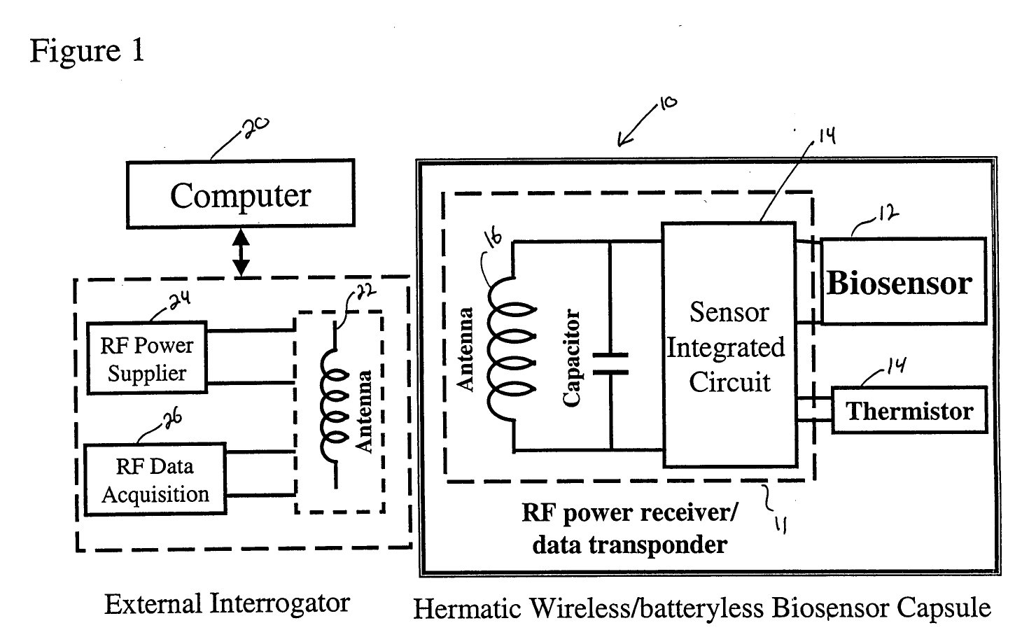

FIG. 1 is a schematic illustration of an implantable capsule 10. Implantable capsule 10 includes one or more biosensors 12 and may include various other measurement devices such as thermistor 14 to take independent measurements or act in concert with biosensor 10. Biosensor 12 may be any type of biosensor including an amperometric, potentiometric, and/or bioimpedance sensor. Capsule 10 is implantable within a human or animal, preferably subcutaneously, in order to measure certain parameters. For example, biosensor 12 may measure and/or detect oxygen saturation within blood, glucose levels, lactate, potassium, protein or various other substances. Capsule 10 is a self-contained unit that includes an integrated circuit to operate the biosensor 12, process the information and transmit that information via antenna 16 to an external interrogator 18. External interrogator 18 may utilize the information itself or may pass the information to another external device such as computer 20.

In order to minimize the size of capsule 10 and allow convenient implantation, no internal power source is included within capsule 10. External interrogator 18 is placed proximate the capsule 10 after implantation. RF energy is transmitted from RF power supply 24 through antenna 22 to the capsule 10 and illuminates the biosensor. The power incident thereon is rectified to produce a DC current to power the IC 14, the biosensor 12, and any other included components. In order to rectify the power, capsule 10 includes RF power receiver 11, which includes components of the IC 14. Of course, other types of energy could be directed towards capsule 10 to deliver power.

The IC 14 modulates backscatter from the antenna 16 based on data collected from the biosensor. This modulated signal is received by antenna 22, demodulated and processed through an RF data acquisition module 26 for subsequent use.

Thus, capsule 10 can be implanted at a desired location. When desired, external interrogator is properly positioned and delivers power to capsule 10. Biosensor 12 and IC 14 receive a DC power supply and function to collect data. For example, biosensor 12 may be a glucose sensor. Thus, after receiving power biosensor 12 measures glucose levels in blood within the tissue surrounding capsule 10.

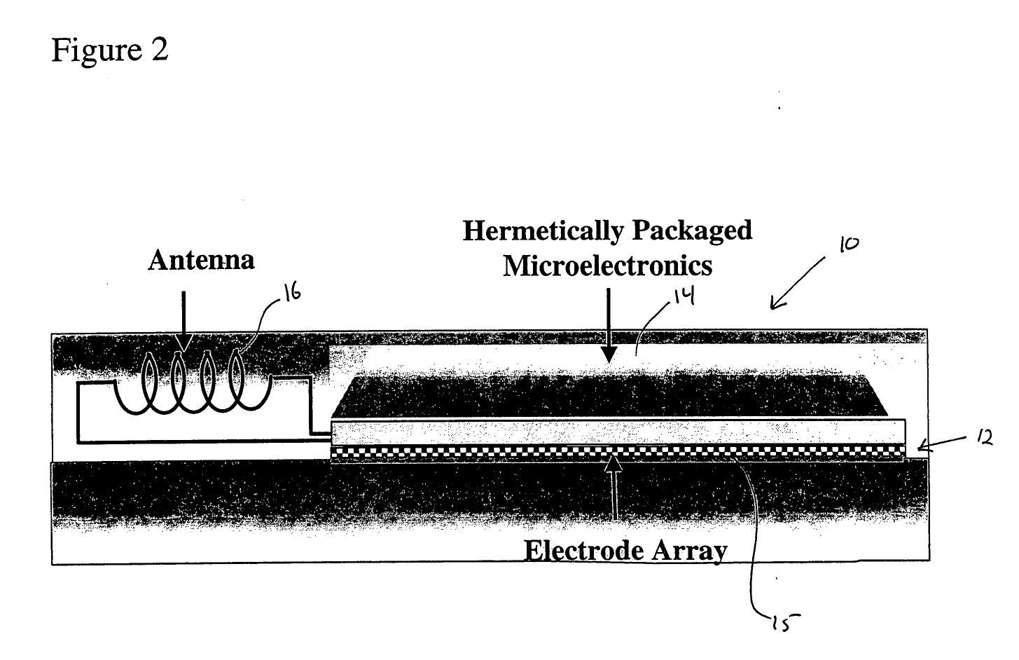

FIG. 2 schematically illustrates capsule 10. The capsule 10 can be made relatively small by eliminating the need for an internal power supply. Thus, the IC 14, biosensor 12 and antenna 16 can be encapsulated and delivered to a desired implant site.

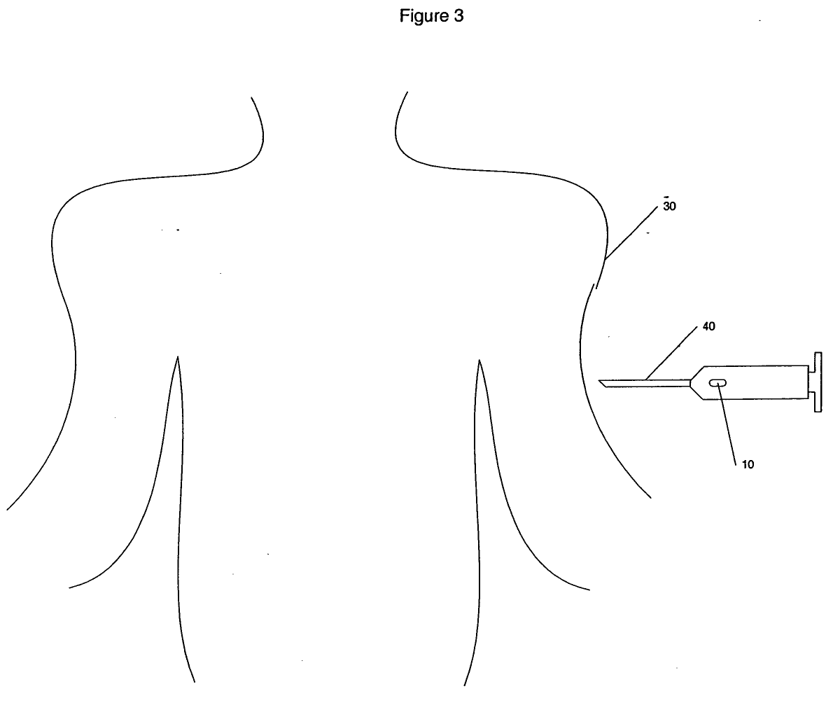

FIG. 3 is a stylized illustration of a human form 30 and syringe 40 useful for implanting capsule 10. Capsule 10 can be implanted subcutaneously or within an artery, vein or other location within the body so long as the location is determinable. That is, since capsule 10 does not contain an internal power supply and instead relies on external power delivery, the location of capsule 10 within body 30 must be determinable. For a subcutaneous implantation, the location is easily determinable as the capsule 10 will not migrate significantly from the implantation site. Furthermore, subcutaneous implantation positions the capsule relatively close to the surface of the tissue. Thus, RF power transmission and data telemetry will have a minimal amount of tissue to pass through.

The capsule 10 is injected subcutaneously into tissue at a desired location. The syringe 40 delivers the capsule 10, optionally along with a small quantity of an inert liquid, such as saline, to facilitate the delivery. Alternatively, any catheter or insertion mechanism could be used to deliver the capsule 10 (alone or in a fluid medium) to a subcutaneous location or to another desired implantation location within the body 30.

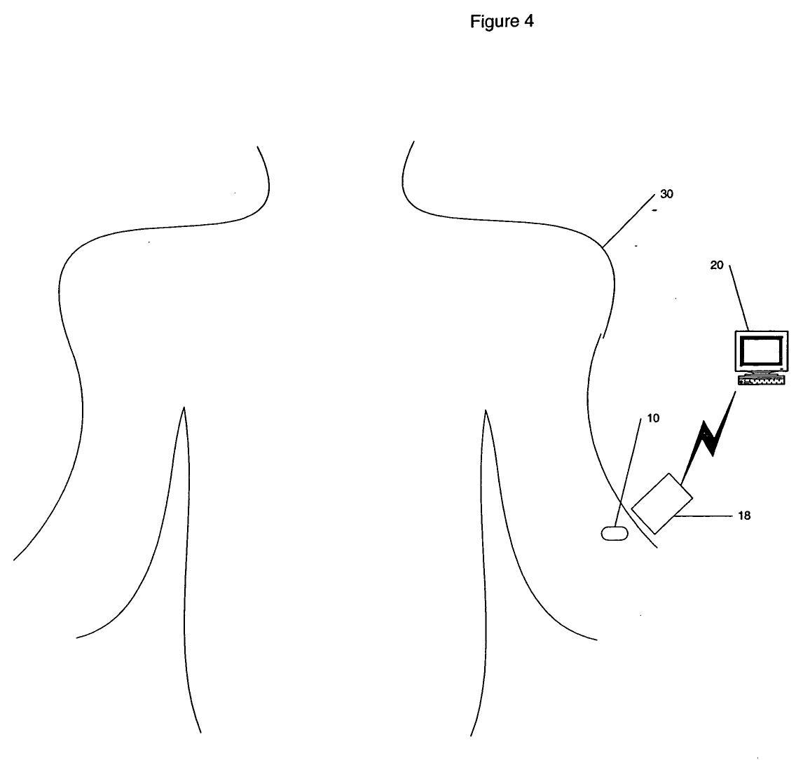

FIG. 4 illustrates the capsule 10 disposed subcutaneously within the body 30. At any desired time, the external interrogator 18 is positioned proximate the known location of the capsule 10. After actuation of the external interrogator 18, RF transmissions from the interrogator 18 pass through the tissue and strike the capsule 10, causing the IC 14 and biosensor 12 contained therein to receive the RF transmissions. The backscatter is rectified into a DC signal and is used to power the IC 14 and the biosensor 12. Biosensor 12 includes an appropriate portion in contact with the surrounding tissue and/or fluid. For example, as illustrated in FIG. 2 an electrode array 15 is provided. Once the DC power is provided, biosensor 12 acts to interface with the biological component of interest. For example, if biosensor 12 is a glucose sensor electrode array 15 may react with glucose to generate hydrogen peroxide, which is electrochemically sensed and generates a quantifiable change in a measurable potential. This is ultimately an indication of the quantity of glucose present. The data so obtained may then be used as desired.

The interrogator 18 is then withdrawn away from the capsule 10, thus terminating the power supplied to the capsule 10. The capsule 10 and the biosensor 12 deactivate. The capsule 10 can then be reactivated and reused with a useful lifetime based on the type of biosensor 12 that is employed. For example, certain biosensors 12 may degrade over time due to contact with tissue or fluids. Other may remain intact indefinitely. As there is no reliance on an internal power supply, the capsule 10 can be relied on for the entire life of the biosensor. Because of its small size and ease of implantation, a new capsule 10 can easily be implanted in order to replace another.

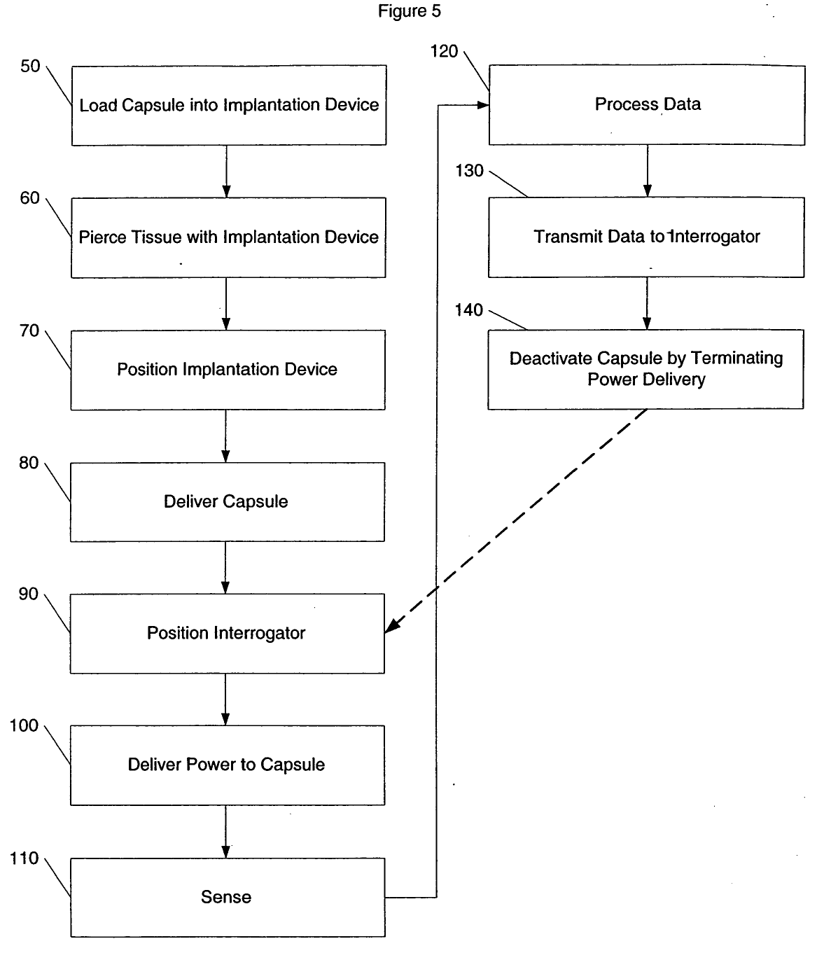

FIG. 5 is a flowchart illustrating a process for implanting and utilizing the capsule 10 containing the biosensor 14. Initially, the capsule 10 is loaded (50) into an implantation device. The device could be a syringe 40 or other or catheter type device. Depending upon the implantation device, the capsule 10 may be loaded before or after the implantation device is positioned within tissue.

The implantation device pierces the tissue (60) at an appropriate location and the implantation device is delivered (70) to the appropriate subcutaneous location. If the capsule is to be implanted at a more remote location, e.g., within a chamber of the heart, the implantation device is delivered to that location. Once properly positioned, the capsule is delivered (80). For example, the syringe may contain a fluid medium (e.g. saline) that is forced into the tissue, carrying the capsule into the implant site. The implantation device is withdrawn and if necessary, any wound created is addressed. At this point, the capsule has been implanted.

When data collection is desired, the interrogator is positioned (90) adjacent to the capsule. That is, the capsule is positioned subcutaneously in a known location. The interrogator is placed near or against the skin most proximate the implantation site. By actuating the interrogator, power is delivered (100) to the capsule 10, thus enabling the capsule and its included biosensor to function. As such, the biosensor is activated and senses (110) the appropriate parameters which are measured by some physical parameter, e.g., a potential, current flow, temperature, or the like. These parameters are processed into a data form (120) and transmitted (130) to the interrogator. The data is utilized in its present form or further processed, if required. The capsule is then deactivated (140) by withdrawing the interrogator, which is the only power source for the capsule. If desired, the process can be subsequently repeated by again positioning the interrogator (90), as previously described.

Although the present invention has been described with reference to preferred embodiments, persons skilled in the art will recognize that changes may be made in form and detail without departing from the spirit and scope of the invention.

1. An implantable sensor comprising:
a biosensor;
an integrated circuit operatively coupled with the biosensor to operate and receive data from the biosensor; and
a power receiver operatively coupled with the integrated circuit and configured to rectify RF energy incident on the implantable senor into DC power deliverable to the biosensor and the integrated circuit.

2. The implantable sensor of claim 1, further comprising:
an antenna coupled to the integrated circuit wherein the integrated circuit modulates data output from the biosensor into a signal and transmits the signal through the antenna.

3. The implantable sensor of claim 2, further comprising:
an external interrogator physically remote from the biosensor and integrated circuit, wherein the interrogator includes an RF power source for transmitting the RF energy to the integrated circuit and the biosensor.

4. The implantable sensor of claim 3, wherein the external interrogator includes a data acquisition module for receiving the modulated signal transmitted from the integrated circuit.

5. The implantable sensor of claim 1, wherein the biosensor is a glucose sensor.

6. The implantable sensor of claim 1, wherein the biosensor measures partial pressure of oxygen.

7. The implantable sensor of claim 1, wherein the biosensor measures pH.

8. The implantable sensor of claim 1, wherein the biosensor measures lactate.

9. The implantable sensor of claim 1, wherein the biosensor measures potassium.

10. The implantable sensor of claim 1, wherein the biosensor detects the presence of a protein.

11. An implantable sensor comprising:
a biosensor;
means for controlling the biosensor; and
means for receiving RF energy from an external source, converting the energy to DC power, and powering the implantable sensor.

12. A method comprising:
injecting a capsule containing an un-powered biosensor subcutaneously into tissue;
placing an interrogator adjacent the tissue;
transmitting RF energy towards the capsule;
converting the RF energy into a DC power source within the capsule; and
utilizing the DC power source to power biosensor.

13. The method of claim 12, further comprising:
transmitting data from the capsule to the interrogator.

14. The method of claim 13, further comprising modulating the data within the capsule prior to transmitting.

15. The method of claim 12, wherein injecting the capsule include inserting the capsule into a syringe and delivering the capsule through the syringe.

16. The method of claim 12, wherein injecting includes delivering the capsule into a coronary sinus of a heart.

17. The method of claim 12, wherein injecting includes delivering the capsule into a right ventricle of a heart
